(19) United States
(12) Patent Application Publication
(10) Pub. No.: US 2025/0143590 A1
(43) Pub. Date: May 8, 2025

(54) CONTINUOUS NONINVASIVE BLOOD PRESSURE MONITORING

(71) Applicant: VITAL SIGNALS, INC., Los Altos, CA (US)

(72) Inventors: Mohammad USMAN, Mission Viejo, CA (US); Faisal KASHIF, Irvine, CA (US)

(21) Appl. No.: 18/937,625

(22) Filed: Nov. 5, 2024

Related U.S. Application Data
(60) Provisional application No. 63/547,582, filed on Nov. 7, 2023.

(51) Int. Cl. A61B 5/021; A61B 5/00; A61B 5/02; A61B 5/024

(52) U.S. Cl. CPC A61B 5/021; A61B 5/02007; A61B 5/742; A61B 5/746; A61B 5/02416; A61B 5/6824; A61B 5/6826

(57) ABSTRACT

Examples include a computing device that receives sensor data from at least one of a photoplethysmographic (PPG) sensor, an electrocardiogram (ECG) sensor, a ballistocardiogram (BCG) sensor, or a microphone. The computing device may input the received sensor data into at least one computational model. The computing device receives an output of the at least one computational model indicative of a blood pressure measurement of a subject. The computing device performs at least one action based on the indicated blood pressure measurement.

### CROSS-REFERENCE TO RELATED APPLICATION

This application claims the benefit of U.S. Provisional Patent Application No. 63/547,582, filed Nov. 7, 2023, which is incorporated by reference herein in its entirety.

### BACKGROUND

Blood pressure may provide an indication of the health of an individual. Conventional techniques for measuring blood pressure typically require the use of an inflatable cuff that is placed on the upper arm or other body part of the individual whose blood pressure is being measured. While the use of an inflatable cuff can usually provide an accurate measurement of an individual's blood pressure, this measurement technique is not practical for frequent and non-intrusive measurement of blood pressure, such as while the individual is going about daily activities, sleeping, or the like.

### BRIEF DESCRIPTION OF THE DRAWINGS

The detailed description is set forth with reference to the accompanying figures. In the figures, the leftmost digit(s) of a reference number identifies the figure in which the reference number first appears. The use of the same reference numbers in different figures indicates similar or identical items or features.

FIG. 1 illustrates an example system for continual, noninvasive, and cuffless blood pressure measurement according to some implementations.

FIG. 2 illustrates an example circuit model that can be used, at least in part, for determining a blood pressure value and/or other physiological characteristics according to some implementations.

FIG. 3 illustrates an example circuit model that can be used, at least in part, for determining a blood pressure value and/or other physiological characteristics according to some implementations.

FIG. 4 illustrates an example arterial pulse waveform that may correspond to a pulse wave measured using a photoplethysmographic (PPG) sensor according to some implementations.

FIG. 5 illustrates an example process for generating a computational model for noninvasive estimation of arterial blood pressure (ABP) according to some implementations.

FIG. 6 illustrates an example of signal processing steps that may be performed for received sensor data according to some implementations.

FIG. 7 illustrates an example of an optimized joint model that may be used to estimate blood pressure based on receiving sensor data as input.

FIG. 8 illustrates an example technique of using a plurality of optimized computational models for determining a blood pressure estimate according to some implementations.

FIG. 9 illustrates an example wearable sensor according to some implementations.

FIG. 10 illustrates an example wearable sensor according to some implementations.

FIG. 11 illustrates an example process for determining a blood pressure value using a joint computational model according to some implementations.

FIG. 12 illustrates an example process for determining a blood pressure value according to some implementations.

FIG. 13 illustrates select example components of a wearable computing device that may implement the functionality described herein according to some examples.

FIG. 14 illustrates select example components of a mobile computing device that may implement the functionality described herein according to some examples.

### DETAILED DESCRIPTION

Some examples herein are directed to techniques and arrangements for blood pressure measurement and monitoring. For instance, some implementations herein may include one or more wearable devices having one or more sensors configured to measure multiple signals at one or more sites on a person's body. As one example, multiple sensing modalities may be employed, such as via a photoplethysmographic (PPG) sensor, an electrocardiogram (ECG) sensor, a ballistocardiogram (BCG) sensor, an inertial measurement unit (IMU), a microphone (MIC) and/or, in some cases, other sensors, as discussed additionally below. In some examples, the BCG sensor may be an accelerometer. In other examples, the IMU may also serve as the BCG sensor while also having the ability to detect other physiological functions such as breathing, activity or position of the individual, and the like.

Additionally, the examples herein may employ one or a plurality of computational models. The computational models herein may include one or more of neural networks or other machine learning models, mathematical models, statistical models, heuristic models, simulation models, combinations thereof, or any of various other types of models that may be used to estimate, calculate, or otherwise assist in determining the blood pressure or other vascular characteristics of a person. Further, while examples herein are described in the context of determining blood pressure measurements for people, the apparatuses, processes, and products herein may be similarly applied to other animals. In some examples, the computational model or models may be obtained from physiological principles and/or may be trained using machine learning methods. The computational models may be configured to provide an indication of blood pressure based on receiving an input of sensor data from one or more sensors. Some examples herein may take into account the anatomy and physiology of the individual, including arterial stiffness and other physical conditions. For instance, the models herein may account for arterial and venous characteristics such as resistance, compliance, and inductance, and may be based in part on physiology for fluid flow and pressure. On the other hand, many conventional systems ignore the differences in the vasculature conditions of individuals, which may vary significantly from one person to the next. In addition, some examples herein may take into account variations in the condition of each individual over time, such as may be due to exercise, medication, hydration level, and so forth.

In some implementations, a single computational model may be trained as a joint model that is able to receive inputs of sensor data from multiple different sensor types, such as PPG, ECG, BCG, sound, IMU, and so forth. For instance, the joint model may incorporate features and/or parameters for all of the different sensor types of which sensor data may be received, and may be optimized according to an estimation template that may be applied in one step on all of the inputted sensor data for determining an estimated blood pressure.

As another example, multiple computational models, such as machine learning models, may be trained, with each respective computational model being trained or derived for a particular sensor type. Under this technique, the system may include an optimized PPG model, an optimized ECG model, an optimized BCG model, and so forth. The outputs of the multiple computational models may be aggregated, such as based on a bias versus estimator variance, for providing an estimated blood pressure of the individual.

In some cases, the estimated blood pressure may be continuously or periodically determined and presented to a user, such as on a display device accessible to the user. In other cases, the estimated blood pressure may be determined and presented on demand, such as in response to a user input. The user may be a person whose blood pressure is being measured or other user, such as a medical professional or other observer.

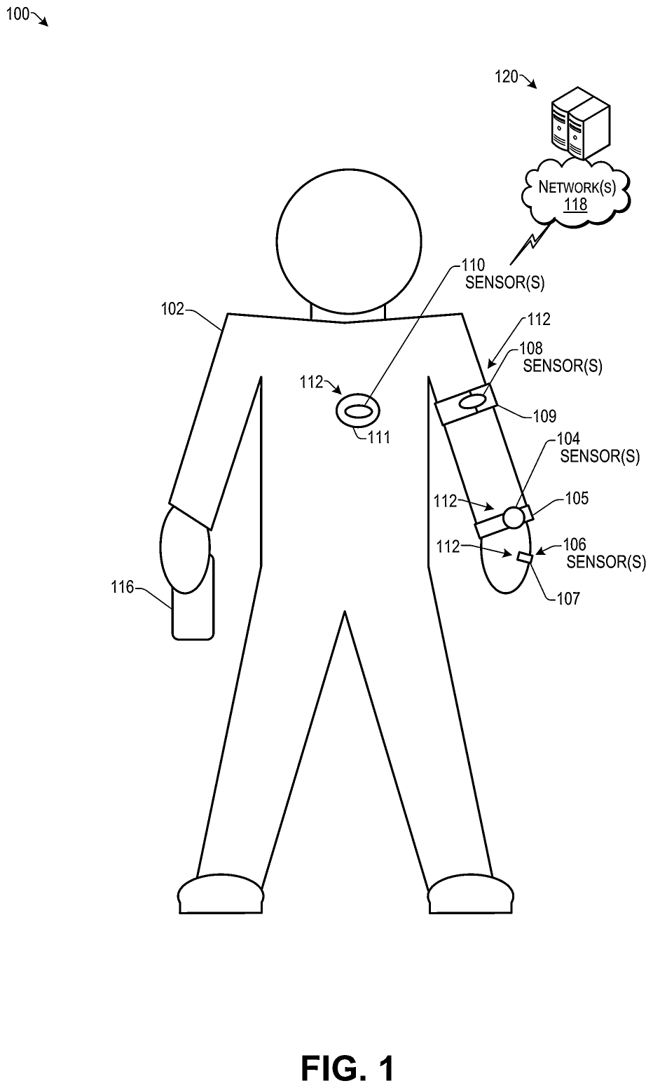

FIG. 1 illustrates an example system 100 for continuous, noninvasive, and cuffless blood pressure measurement according to some implementations. In this example, a person 102 may wear or may otherwise be in contact with one or more sensors, such as one or more sensors 104 included on a wrist-mountable device 105, such as a watch, bracelet, or the like, mounted on the wrist of the person 102; one or more sensors 106 included on a finger-mountable device 107, such as a ring, fingertip-worn device, artificial fingernail, or the like, mounted on one or more fingers/thumbs of the person 102; one or more sensors 108 included on an arm-mountable device 109, such as a sleeve, strap, band, patch, or the like, mounted on the upper arm or forearm of the person 102; and/or one or more sensors 110 included on a chest-mountable device 111, such as a patch, strap, belt, harness, or the like, mounted on the chest of the person 102.

Further, while several sensor locations and types of mounting devices are shown and described in this example, not all of these sensor locations and mounting devices need to be used, and/or other sensor locations and types of mounting devices may be used instead, as will be apparent to those of skill in the art having the benefit of the disclosure herein. For instance, in some examples, a single sensor may be employed at a single location on the body of the person 102, such as on the finger, wrist, arm, chest, etc., of the person 102. However, in other examples, the use of multiple sensors of different types and/or at different body locations may help to solve for unknowns and mitigate uncertainty. Further, by combining the outputs of different sensors of different types and/or at different body locations, a more accurate and robust outcome may be achieved.

In some examples, the one or more sensors 104-110 may include a photoplethysmographic (PPG) sensor, an electrocardiogram (ECG) sensor, a ballistocardiogram (BCG) sensor, a microphone, IMU, and/or other sensors. In some implementations, employing multiple sensing modalities at several sites on the body may provide greater accuracy in measuring blood pressure, but at a tradeoff of being more intrusive on the individual. In some cases, use of the computational models herein, such as machine learning models with sufficient training data, may enable a reduction in the number and type of sensors used, as well as enabling reduction in the number of sites on the body at which the sensors are placed. Further, some examples may include a mathematical model for accounting for unknown parameters of the vascular system, such as arterial resistance, compliance, and elastance.

As one example, the finger-mountable device 107, such as a ring, fingertip-worn device, artificial fingernail, or the like, that is mounted on the finger of the person 102 may include at least one sensor (not shown in FIG. 1), such as a PPG sensor, an ECG sensor, a BCG sensor, a sound sensor, an IMU, or the like. As discussed additionally below, in some cases, the finger-mountable device may employ, for blood pressure measurement, only a PPG sensor. For instance, the PPG sensor may include at least a light source and an optical sensor. The light source may generate a beam of light that is detected by the optical sensor, such as by reflecting off a body part of interest (e.g., an artery) or by passing through a body part of interest. In some cases, the PPG sensor may be of higher fidelity than conventional PPG sensors, such as having a sampling rate of 1000-2000 Hz, or even higher. Additionally, in other examples, the finger-mountable device 107 may include a plurality of sensors (not shown in FIG. 1) such as two or more of the PPG sensor, ECG sensor, BCG sensor, sound sensor, IMU, or other type of sensor.

In some examples, the one or more sensors 110 may be employed, and may be included with the chest-mountable device 111 that positions the one or more sensors 110 on the chest of the person 102. For example, the chest-mountable device 111 may include one or more of an ECG sensor, a BCG sensor, a sound sensor, an IMU, and/or an optical sensor and light source able to operate as a PPG sensor. As another example, one or more sensors 108 such as an ECG sensor, a BCG sensor, a sound sensor, an IMU, and/or an optical sensor and light source able to operate as a PPG sensor may be mounted on the upper arm or forearm of the person 102, such as on the arm-mountable device 109. As still another example, one or more sensors 104 such as an ECG sensor, a BCG sensor, a sound sensor, an IMU, and/or an optical sensor and light source able to operate as a PPG sensor may be mounted on the wrist of the person 102, such on the wrist-mountable device 105. Sensor data collected by the one or more sensors 104-110 herein may include an ECG, a track volume, a heart rate, a respiration rate, motion, sound, mechanical recoil, and the like.

In some cases, the one or more sensors 104-110 may be associated with a wearable computing device 112 that may include at least one of a human-machine interface for communicating information to a human, and/or an electronic communication capability for communicating electronically with other computing devices. For instance, any of the body-mountable devices discussed above, such as the finger-mountable device 107, the wrist-mountable device 105, the arm-mountable device 109 and/or the chest-mountable device 111 may include a built-in wearable computing device 112. In some cases, each of the sensors 104-110 may provide sensor data to the wearable computing device 112. The wearable computing device 112 may include at least one processor for processing the sensor data to determine a blood pressure of the person 102, as discussed additionally below. For instance, the wearable computing device 112 may include a blood-pressure-determining functionality for continually determining the blood pressure of the person 102 over a period of time based at least in part on the sensor data received from one or more of the sensors 104-110.

The wearable computing device 112 may present, via the human-machine interface, one or more determined blood pressure (BP) values. As one example, the human-machine interface may include a display associated with the wearable computing device 112 to display the BP value(s), and/or may include a speaker that produces sound to announce the BP value(s), or the like.

In some cases, each of the one or more sensors 104-110 may provide sensor data to a single wearable computing device 112. Alternatively, in other examples, each of the sensors 104-110 may each be associated with a separate wearable computing device 112 that is able to at least communicate the respective sensor data to another computing device. As one example, each of the one or more sensors 106-110 may communicate wirelessly or through a wired connection with a wearable computing device 112 included with the wrist-mountable device 105 to provide sensor data to the wearable computing device 112 included with the wrist-mountable device 105, which may then perform processing to present a blood pressure value on a display or the like. Furthermore, while the wearable computing device 112 is described as being wrist-mountable in this example, in other examples, the wearable computing device 112 mounted at any of the sensor locations of the sensors 104-110 or, alternatively, at a different location on the body of the person 102, may include a wearable computing device 112 that receives sensor data from the other one or more sensors 104-110 for determining a blood pressure (BP) value based on the techniques described below.

As another alternative, each of the wearable computing devices 112 may perform its own processing to determine a local BP value based on local (on-board) sensor data to determine a local BP value, and may transmit this local BP value to a primary one of the wearable computing devices 112, such as the wearable computing device 112 included on the wrist-mountable device 105. The wearable computing device 112 on the wrist-mountable device 105 may also determine a local BP value from the sensor(s) 104, and may then determine one or more BP values to present on an associated display. As several examples, the presented BP value may be an average of the received BP values and the local BP value, or may be a weighted BP value, such as by according more weight to BP values determined by devices 105, 107, 109 or 111 that include a larger number of sensors. As yet another example, the primary wearable computing device 112 on the wrist-mountable device 105 may present some or all of the received BP values and the locally determined BP value, such as along with an indication of the body location at which the measurement was taken. Other variations will be apparent to those of skill in the art having the benefit of the disclosure herein.

Additionally, or alternatively, in some examples, the wearable computing device 112 may communicate through wireless and/or wired communication with a mobile computing device 116 associated with the person 102. For instance, the mobile computing device 116 may be a smartphone, tablet, or the like, and may include the functionality for determining the blood pressure of the person 102 instead of, or in addition to, the wearable computing device(s) 112. For instance, similar to the examples discussed above, each of the wearable computing devices 112 may transmit the sensor data from the one or more sensors 104-110 to the mobile computing device 116, which may then perform processing to determine a BP value based on the received sensor data using the techniques described below. Alternatively, each of the wearable computing devices 112 may perform its own processing to determine a local BP value based on local (on-board) sensor data to determine a local BP value, and may transmit this local BP value to the mobile computing device 116, which may then determine one or more BP values to present on an associated display of the mobile computing device 116 or on display(s) of one or more of the wearable computing devices 112.

Furthermore, in some examples, the wearable computing devices 112 may be omitted, and the one or more sensors 104-110 may communicate directly with the mobile computing device 116 through wireless and/or wired communication for providing sensor data directly to the mobile computing device 116. In this example, the mobile computing device 116 may present the BP value(s) on a display associated with the mobile computing device 116, and/or may provide an audible indication of the BP value(s), or the like.

Additionally, or alternatively, the wearable computing device(s) 112, the mobile computing device 116, and/or the one or more sensors 104-110 themselves may be able to communicate over one or more networks 118 with one or more network computing devices 120, such as one or more cloud servers, one or more service computing devices, one or more local computing devices, or the like. Further, in some implementations, the one or more network computing devices 120 may send, to at least one of the mobile computing device 116 or the wearable computing device(s) 112, one or more computational models, such as machine learning models (not shown in FIG. 1), that may be periodically updated based on training data, and used for determining, at least in part, the BP value(s) based at least in part on received sensor data from one or more of the sensors 104-110. The one or more networks 118 may include any suitable type of communication network enabling wireless and/or wired communications, and may include local area networks, wide area networks, the Internet, virtual private networks, cellular networks, WiFi networks, close range communications, such as BLUETOOTH®, and so forth.

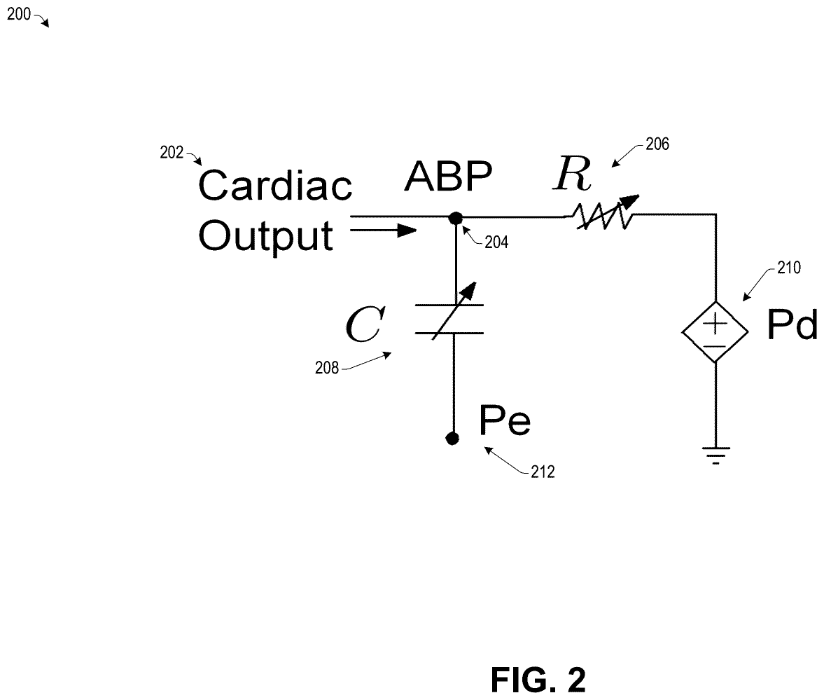

FIG. 2 illustrates an example circuit model 200 that can be used, at least in part, for determining a BP value and/or other physiological characteristics according to some implementations. For instance, the circuit model 200 may be correlated to physiological characteristics of a person's vascular system for calculating values of blood pressure and/or other physiological characteristics, such as vascular resistance and/or vascular compliance for a portion of a person's vascular system. In this example, as indicated at 202, cardiac output (e.g., an incoming flow to a portion of the vascular system) may be received as an input to the circuit model 200 for determining the arterial blood pressure (ABP), as indicated at 204. As one example, the cardiac output 202 corresponding to the incoming flow may be measured using one or more of the sensors 104-110 discussed above with respect to FIG. 1. The circuit model 200 in this example further includes a variable resistance R, as indicated at 206, which may be correlated to vascular resistance; a variable capacitance C, as indicated at 208, which may be correlated to vascular compliance; a controlled voltage source, as indicated at 210, which may be correlated to the arterial blood pressure Pd; and a voltage, as indicated at 212, which may be correlated to an external pressure Pe.

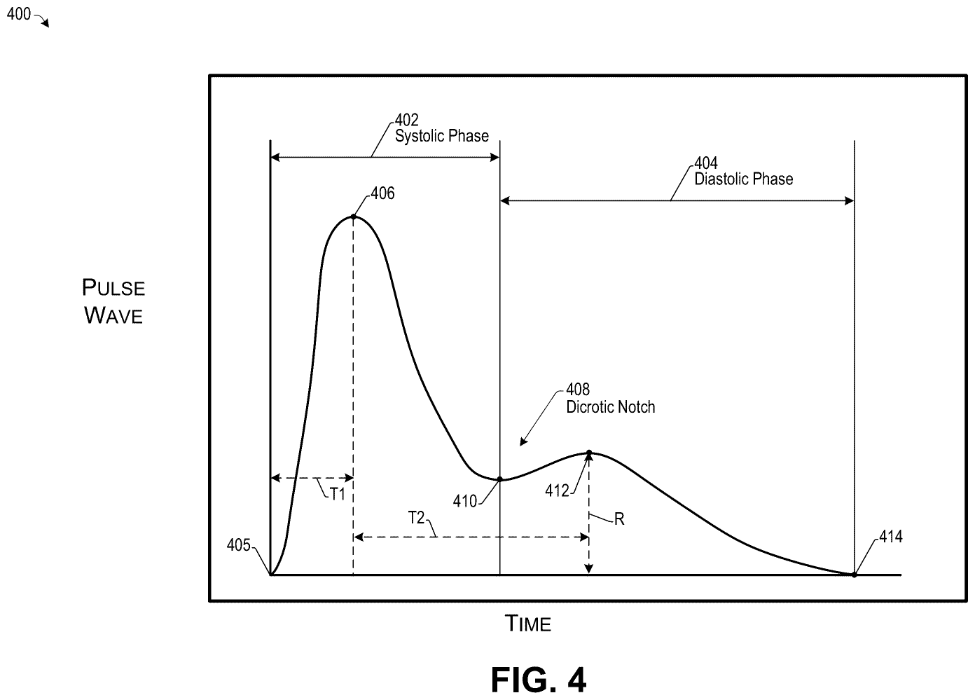

As one example, the circuit model 200 may initially be used to determine values for the compliance and resistance of a portion of an individual's vascular system, such as based in part on measurement of the flow (cardiac output) at 202 and based on some knowledge of the external pressure Pe and/or some knowledge of the phase of the waveform of the flow (cardiac output) at 202, such as is illustrated and discussed additionally below, e.g., with respect to FIG. 4. For instance, during some segments of the waveform, R and C may be sufficiently decoupled from each other to make it possible to solve for one or the other. Once values for compliance and resistance have been determined for the individual, these values may then be used for calculating the blood pressure Pd of the individual based on received sensor data received from the one or more sensors 104-110 over time. Thus, implementations herein enable consideration of the physiology of an individual when calculating the blood pressure from sensor data.

The circuit model 200 may be used to provide constraints for the pressure and flow through the arteries of an individual whose blood pressure is being monitored. As mentioned above, the circuit model 200 may also capture at least some physical properties of the vasculature of the individual, and may represent these physical properties as lumped compliance and/or resistance in some cases based on analogy to the capacitance at 208 indicating vascular compliance, and the resistance at 206 indicating vascular resistance, respectively, in the model 200. Furthermore, spatially different segments of an artery can be represented by separate circuit models 200 of multiple circuit models 200 that may be arranged in series for each of the different segments of the artery, respectively, for determining physiology of the corresponding artery segments The model 200 may be used to guide the development of a computational algorithm for use in processing the measurements captured by the sensors discussed above.

In some cases, the calculated resistance and compliance may be used to calculate the blood pressure Pd directly. Additionally or alternatively, in some examples, the compliance and/or resistance determined using the circuit model 200 may be used as inputs and/or features of the computational model(s) herein, and/or may be used as markers, etc. As yet another example, the resistance and compliance may change over time based on some changes in the state of the individual, such as due to medication, exercise, or the like. In this situation, different techniques may be applied for determining the blood pressure of the individual based on the current state of the individual.

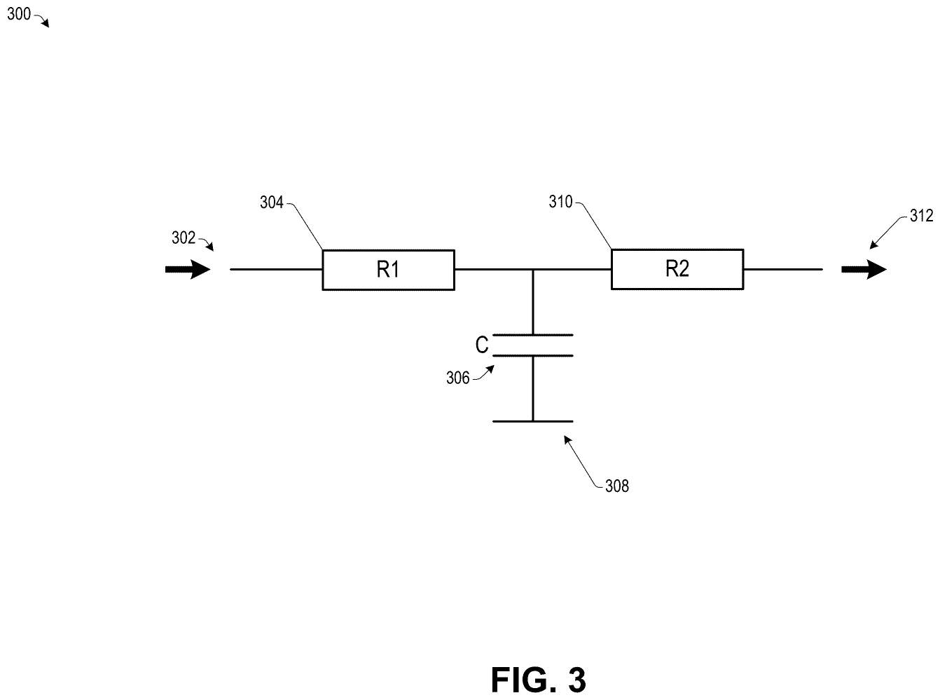

FIG. 3 illustrates an example circuit model 300 that can be used, at least in part, to determine a BP value and/or other physiological characteristics according to some implementations. For instance, the circuit model 300 (or yet other circuit models—not shown in FIG. 3) may be better suited for different sensor types, different sensor locations, and/or different vascular physiologies. In this example, the circuit model 300 includes an input 302 (e.g., a flow), a first resistance R1, as indicated at 304, a capacitance C, as indicated at 306, having a ground plane 308, a second resistance R2, as indicated at 310, and an output 312. For example, the circuit model 300 may be a better model in a situation in which there is some resistance upstream of the intended target vascular portion, such as in the situation in which the arterial resistance varies significantly along the length of an artery.

FIG. 4 illustrates an example arterial pulse waveform 400 that may correspond to a pulse wave measured using a photoplethysmographic (PPG) sensor according to some implementations. The waveform 400 is plotted as a function of pulse wave amplitude versus time. In this example, the waveform 400 includes a systolic phase 402 and a diastolic phase 404. During the systolic phase 402, the systolic phase begins at 405, and a systolic peak occurs at 406 followed by a dicrotic notch 408 including a dicrotic trough 410 that may correspond to closing of the aortic valve and/or reflected pressure waves. The dicrotic trough 410 indicates the end of the systolic phase 402 and the beginning of the diastolic phase 404. Furthermore, a diastolic peak occurs at 412 during the diastolic phase 404. The end of the diastolic phase at 414 indicates the opening of the aortic valve and the beginning of a next systolic phase.

The shape of the pulse wave, including the amplitudes of the systolic peak 406 and diastolic peak 412, the slopes of the waveform 400, the dicrotic notch size and location, and so forth, may vary significantly from one individual to another, and can provide an indicator of the physiological condition of the vascular system of the individual. Examples of information that may be considered pertinent may include the length of time T1 between the beginning 405 of the systolic phase 402 and the systolic peak 406, the time T2 between the systolic peak 406 and the diastolic peak 412, as well as the height R of the diastolic peak 412 relative to the end point 414. These pieces of information relate to the dynamics of the blood flow and pressure, such as, when the heart goes into systole and then to its diastolic phase. Therefore, physiological relationships can be estimated, such as via an inverse function estimation, e.g., using a regression or a machine-learning approach to determine a mathematical function. For example, some or all of the pieces of information discussed above for this waveform (and/or additional pieces of information) may be used as features in the one or more machine learning models or other computational models disclosed herein.

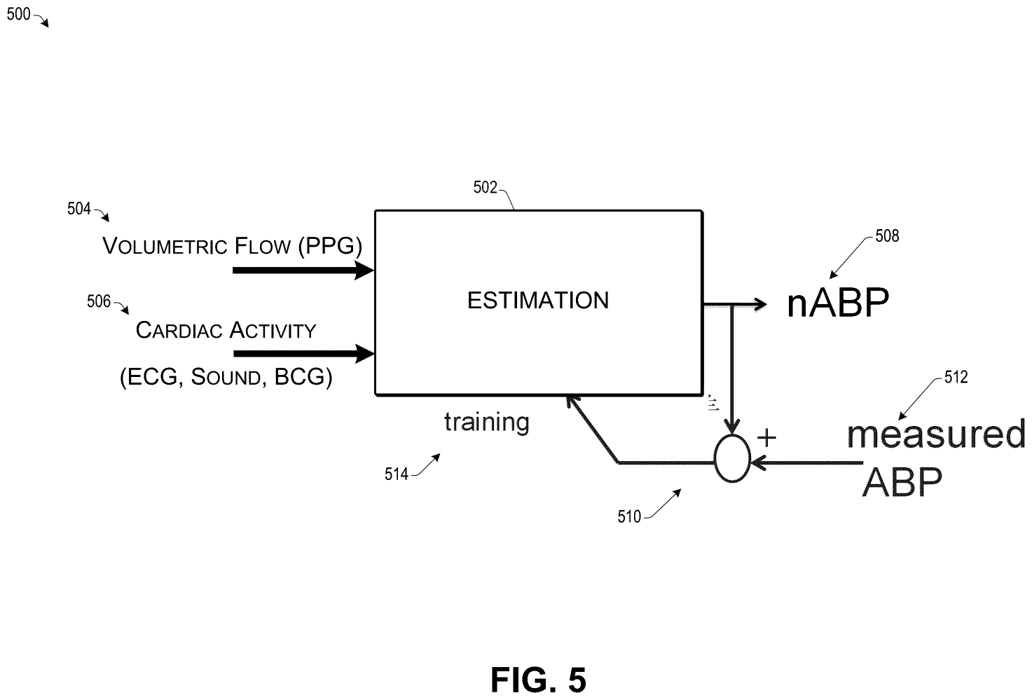

FIG. 5 illustrates an example process 500 for generating a computational model for noninvasive estimation of arterial blood pressure (ABP) according to some implementations. In this example, an estimation model 502 may receive sensor information including volumetric flow 504 such as may be measured by a PPG sensor. The estimation model 502 further receives an indication of cardiac activity 506, such as may be measured using an ECG sensor, a sound sensor, and/or a BCG sensor. The estimation model 502 outputs an estimated arterial blood pressure nABP, as indicated at 508. At 510, the estimated arterial blood pressure nABP is compared with a measured ABP 512, such as may be measured using any of the conventional gold standard techniques, which may include the use of an inflatable cuff in some examples. During training of the estimation model 502, as indicated at 514, the difference between the estimated arterial blood pressure nABP and the measured ABP is used for adjusting one or more parameters of the estimation model 502.

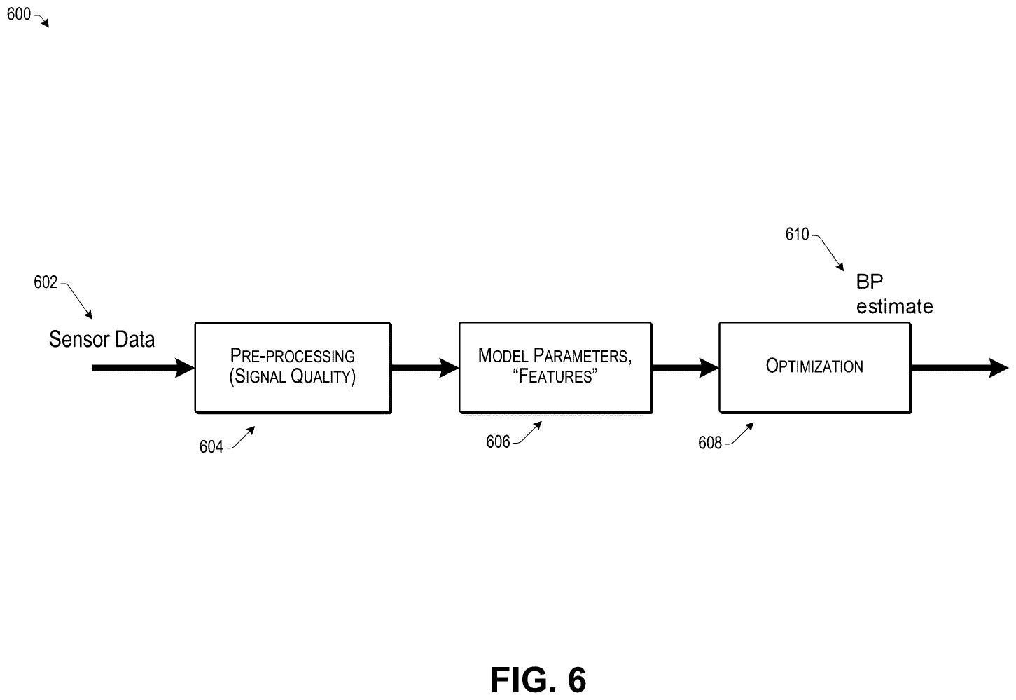

FIG. 6 illustrates an example of signal processing steps 600 that may be performed for received sensor data according to some implementations. In this example, as indicated at 602, sensor data may be received from at least one of the one or more sensors 104-110 discussed above with respect to FIG. 1. At 604, preprocessing is performed such as for improving the signal quality of the sensor data. Additionally, at 606, one or more features of the sensor data are identified for use as one or more corresponding model parameters of an estimation model. At 608, the model parameters are used for estimating a corresponding blood pressure from the sensor data as indicated at 610, and optimization of the estimation model may be performed, such as discussed above with respect to FIG. 5.

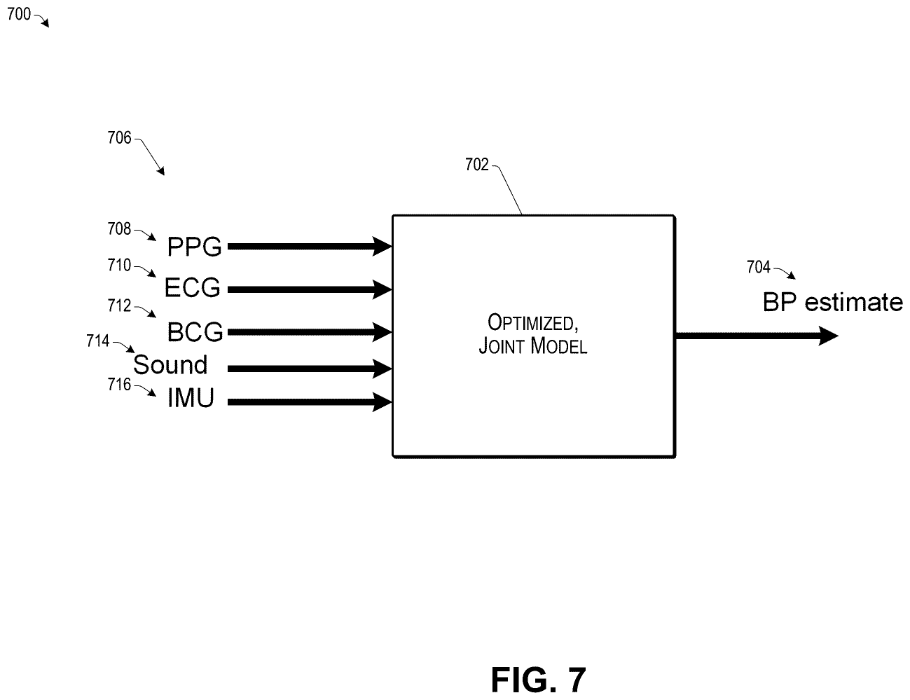

FIG. 7 illustrates an example of an optimized joint model 702 as a computational model that may be used to estimate blood pressure as indicated at 704 based on receiving sensor data 706 as input. In this example, sensor data 706 received as inputs to the optimized joint model 702 may include PPG sensor data 708, ECG sensor data 710, BCG sensor data 712, sound sensor data 714, and/or IMU sensor data 716. The model 702 may be optimized such as using the process discussed above with respect to FIG. 5.

Examples of machine learning models that may be used as the optimized joint model 700 and the other trainable models described herein may include neural networks such as deep learning models, recurrent neural networks, LSTM models, convolutional neural networks, feedforward neural networks, and so forth, to name a few. Further, other types of machine learning and statistical models may be used, such as models trained using supervised learning algorithms (e.g., Bayesian statistics, support vector machines, decision trees, classifiers, k-nearest neighbor, etc.), unsupervised learning algorithms (e.g., association rule learning, hierarchical clustering, cluster analysis, etc.), semi-supervised learning algorithms, and so forth. Implementations herein are not limited to any particular type of machine learning or otherwise trainable model. Alternatively, in some examples, one or more heuristic models and/or mathematical models, or the like, may be used to perform some or all of the functions described herein in place of one or more machine learning models.

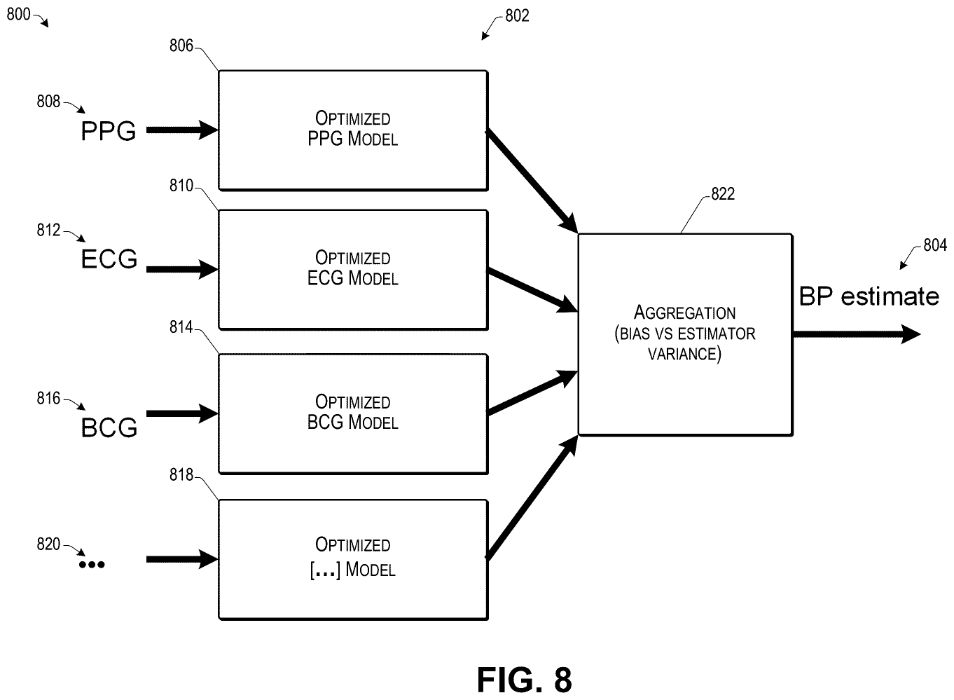

FIG. 8 illustrates an example technique 800 of using a plurality of optimized models 802 as a plurality of computational models for determining a blood pressure estimate 804 according to some implementations. In this example, a respective optimized model is provided for different types of sensor data. Accordingly, an optimized PPG model 806 is employed for receiving PPG sensor data 808 as an input; an optimized ECG model 810 is employed for receiving ECG data 812 as an input; an optimized BCG model 814 is employed for receiving BCG data 816 as an input; and in some examples one or more additional optimized models 818 may be employed for receiving one or more additional different types of sensor data 820, respectively, as inputs.

In this example, each of the models 806, 810, 814, 818 may output a blood pressure estimate that may be aggregated at 822 to provide the output blood pressure estimate 804. As one example, the aggregation may include a biased-versus-estimator variance that provides weighting to the outputs of certain ones of the models 806, 810, 814, 818 as compared to the outputs of other ones of the models 806, 810, 814, 818. As one example, the output of the optimized PPG model 806 may be given greater weight than the output of the optimized BCG model 814.

Additionally, in some cases, a time consideration may be included in the aggregation component 822 that takes into consideration how the blood pressure estimate is changing over time for providing a more accurate estimation of the blood pressure. For example, by looking at consecutive blood pressure estimates, or blood pressure estimates over a small time interval, a confidence metric or stability of estimation can be formed. This may be used to guide the aggregator at 822 to accept or reject a blood pressure estimate. Additionally, the signal quality of the input measurements may vary over time (e.g., due to poor connection or sensor attachment). Therefore, the aggregator may use a technique such as SQA (signal quality assessment) for individual estimators/sensors to judiciously combine these results. For example, the aggregator at 822 may choose to drop a blood pressure estimate during certain bad periods of receiving a poor signal from one or more of the sensors.

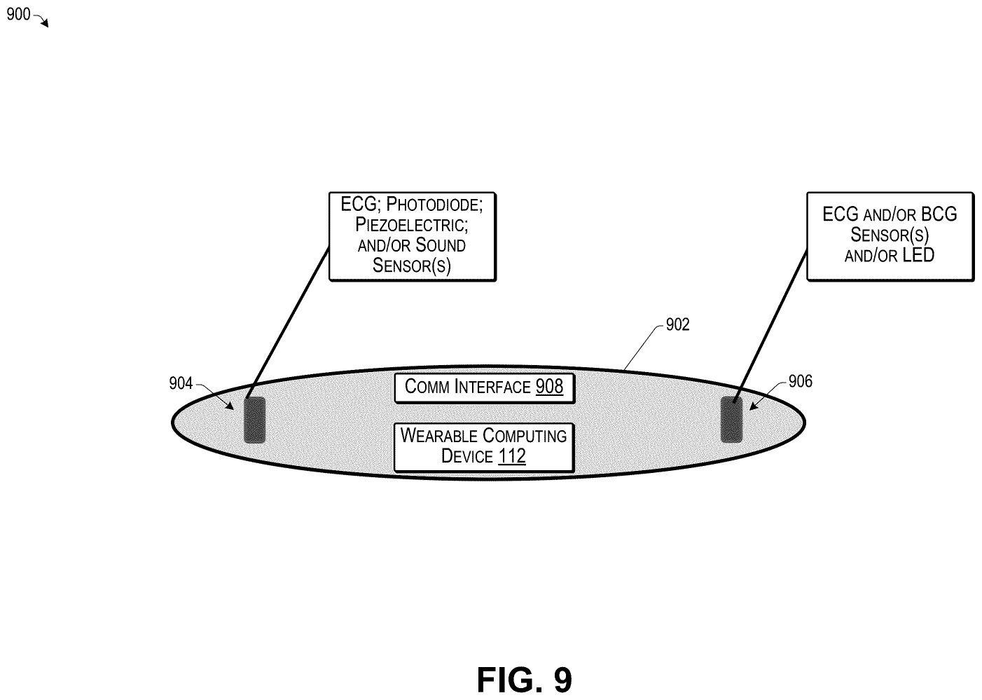

FIG. 9 illustrates an example wearable sensor 900 according to some implementations. In this example, the wearable sensor 900 may be worn on the chest or other body location of an individual, such as in the form of a patch 902 or the like. In the illustrated example, the wearable sensor 900 includes sensors at least at two locations, with a first location 904 including an ECG sensor, a photodiode, a piezoelectric sensor and/or a sound sensor (microphone). Additionally, the second sensor location 906 may include an ECG sensor and/or a BCG sensor, and/or an LED for providing directed light that is detectable by the photodiode at 904 for providing PPG sensor data. In some examples, the BCG sensor may be an accelerometer or an IMU.

The wearable sensor 900 may further include additional components such as a wireless communication interface 908 to enable communication with a computing device such as through BLUETOOTH® or other short range radio signals, a battery (not shown), and so forth. Additionally, in some examples, the wearable sensor 900 may include a wearable computing device 112, which may include at least one processor and memory, and that may perform processing of the received sensor data received from the sensors at 904, 906 (and other sensors at other locations in some examples) for providing an estimated BP value based on the received sensor data.

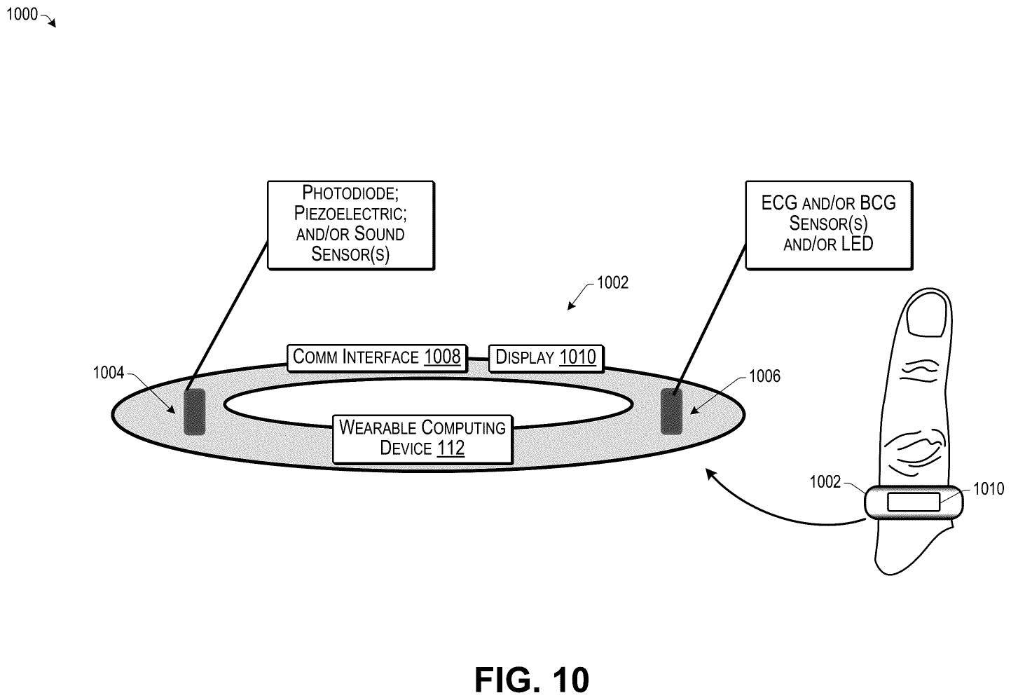

FIG. 10 illustrates an example wearable sensor 1000 according to some implementations. In this example, the wearable sensor 1000 may be a finger-mountable device 1002, such as a ring that may be mounted on a finger or thumb of a user. In other examples, the wearable sensor 1000 may be an arm-mountable device worn on the arm of an individual such as in the form of a strap, band, sleeve, patch, or the like. Alternatively, in other examples, the wearable sensor 1000 may be a wrist-mountable device worn on the wrist of a person, such as in the form of a watch, bracelet, wristband, or the like. As still another alternative, the wearable sensor 1000 may be a different type of finger-mountable device, such as a fingertip-mountable device, an artificial fingernail, a glove that may be worn on the hand of a person, or the like.

The wearable sensor 1000 includes sensors at least at two locations, with a first location 1004 including a photodiode, a piezoelectric sensor and/or a sound sensor (microphone). In this example, the second sensor location 1006 may include an ECG sensor and/or a BCG sensor, and/or an LED for providing directed light that is detectable by the photodiode for providing PPG sensor data. In some examples the BCG sensor may be an accelerometer or an IMU.

Additionally, in some examples, the finger-mountable device 1002, such as a ring, may include only one or more PPG sensors that are used for detecting the blood pressure of a user of the finger-mountable device 102, and the other sensors mentioned above may be omitted. For example, one or more photodiodes and one or more LEDs may be located on an inner circumference of the finger-mountable device 1002 for serving as one or more PPG sensors. Furthermore, in some examples, the PPG sensor(s) may be operated at sampling rate that is substantially higher than conventional PPG sensors, such as at a sampling rate between 1000 and 2000 Hz, or even higher, which provides a high-fidelity detected fluctuations in blood vessel diameters, locations, and so forth. This enables provision of a higher fidelity waveform of this information as the sensor data input signal, which provides for a more accurate measurement.

The wearable sensor 1000 may further include additional components such as a wireless communication interface 1008 to enable communication with a computing device such as through BLUETOOTH® or other short range radio signals, a display 1010, a battery (not shown), and so forth. Additionally, in some examples, the wearable sensor 1000 may include a wearable computing device 112 that may include at least one processor and memory, and that may perform processing of the received sensor data received from the sensors at 1004, 1006 (and/or other sensors at other locations in some examples) for providing an estimated BP value based on the received sensor data.

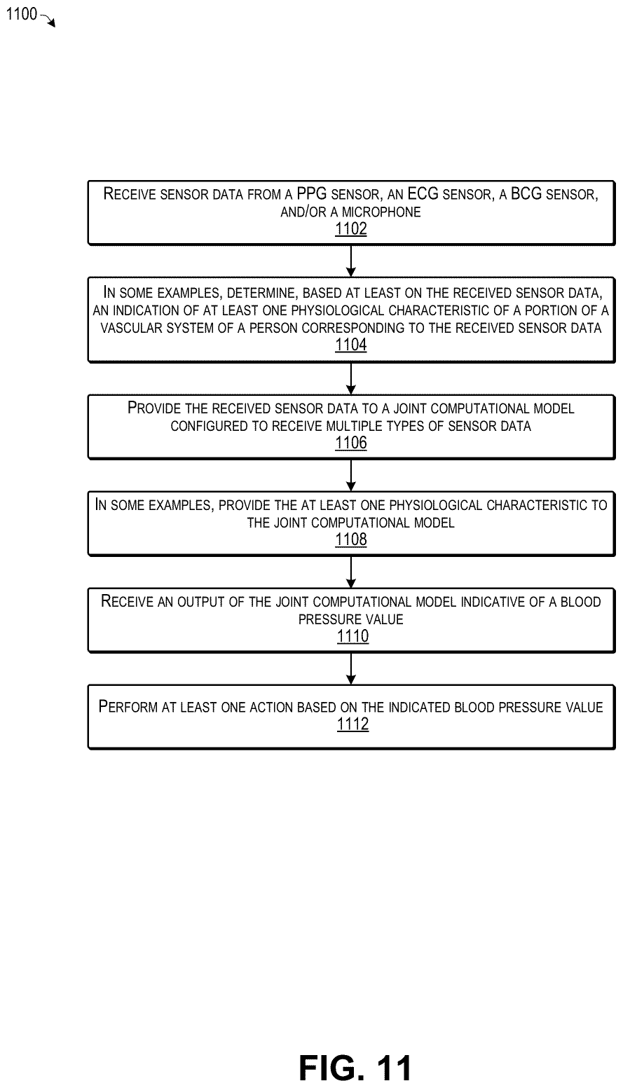

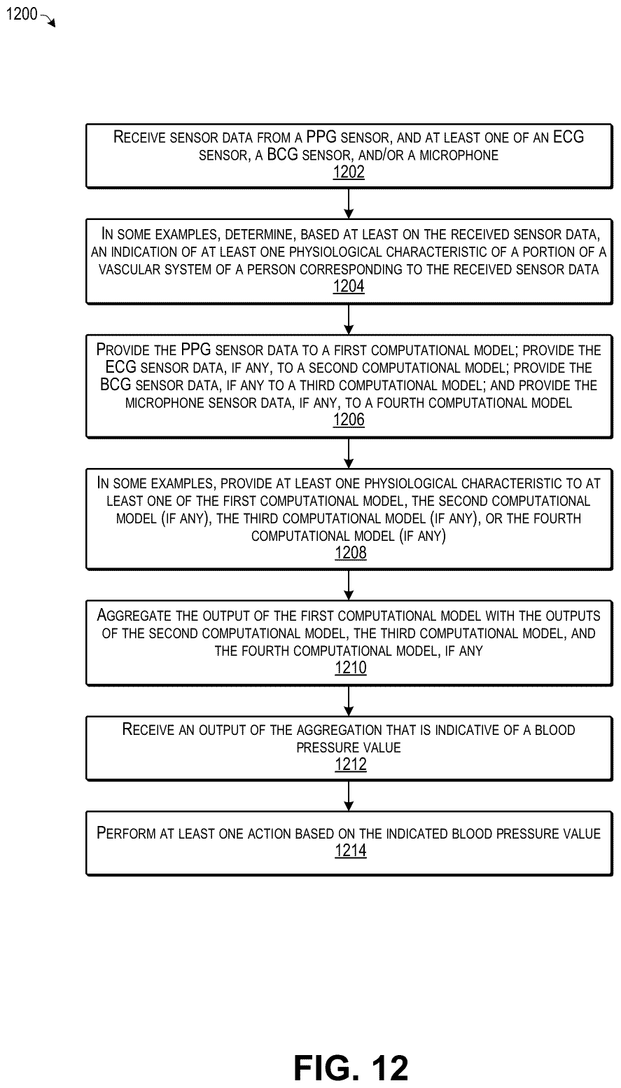

FIGS. 11-12 include flow diagrams illustrating example processes according to some implementations. The processes are illustrated as collections of blocks in logical flow diagrams, which represent a sequence of operations, some or all of which can be implemented in hardware, software or a combination thereof. In the context of software, the blocks may represent computer-executable instructions stored on one or more computer-readable media that, when executed by one or more processors, program the processors to perform the recited operations. Generally, computer-executable instructions include routines, programs, objects, components, data structures and the like that perform particular functions or implement particular data types. The order in which the blocks are described should not be construed as a limitation. Any number of the described blocks can be combined in any order and/or in parallel to implement the process, or alternative processes, and not all of the blocks need be executed. For discussion purposes, the processes are described with reference to the environments, systems and devices described in the examples herein, although the processes may be implemented in a wide variety of other environments, systems and devices.

FIG. 11 illustrates an example process 1100 for determining a BP value using a joint computational model according to some implementations. In some examples, the process 1100 may be executed by a suitable computing device, such as one or more of the wearable computing devices 112 or the mobile computing device 116, as discussed above with respect to FIG. 1, or by any other computing device that may receive the sensor data. For example, the computing device may determine the BP value at least in part by executing a blood pressure program on one or more processors (not shown in FIG. 11).

At 1102, the computing device may receive sensor data from a PPG sensor, an ECG sensor, a BCG sensor, and/or a microphone.

At 1104, in some examples, the computing device may determine, based at least on the received sensor data, an indication of at least one physiological characteristic of a portion of a vascular system of a person corresponding to the received sensor data. For example, the techniques discussed with respect to FIGS. 2 and 3 may be used to determine the physiological characteristics of the subject.

At 1106, the computing device may provide the received sensor data to a joint computational model configured to receive multiple types of sensor data.

At 1108, in some examples, the computing device may provide the at least one physiological characteristic to the joint computational model.

At 1110, the computing device may receive an output of the joint computational model indicative of a BP value.

At 1112, the computing device may perform at least one action based on the indicated BP value. For example, the computing device may initiate an alert, and/or may present the BP value on a display associated with the computing device, e.g., as part of a continuous, periodic, or on-demand display of the user's blood pressure. As another example, the computing device may send the BP value to another device to cause the other device to perform an action, such as initiating an alert, presenting the BP value on a display, performing further processing with the BP value, or the like.

FIG. 12 illustrates an example process 1200 for determining a BP value using a plurality of computational models according to some implementations. In some examples, the process 1200 may be executed by a suitable computing device, such as the wearable computing device(s) 112 or the mobile computing device 116, as discussed above with respect to FIG. 1, or any other computing device that may receive the sensor data. For example, the computing device may determine the BP value at least in part by executing a blood pressure program on one or more processors (not shown in FIG. 12).

At 1202, the computing device may receive sensor data from a PPG sensor, an ECG sensor, a BCG sensor, and/or a microphone.

At 1204, in some examples, the computing device may determine, based at least on the received sensor data, an indication of at least one physiological characteristic of a portion of a vascular system of a person corresponding to the received sensor data. For example, the techniques discussed with respect to FIGS. 2 and 3 may be used to determine the physiological characteristics of the subject.

At 1206, the computing device may provide the PPG sensor data to a first computational model trained to determine a BP value based on receiving PPG sensor data as an input; provide the ECG sensor data, if any, to a second computational trained to determine a BP value based on receiving ECG sensor data as an input; provide the BCG sensor data, if any to a third computational model trained to determine a BP value based on receiving BCG sensor data as an input; and provide the microphone sensor data, if any, to a fourth computational model trained to determine a BP value based on receiving sound sensor data as an input.

At 1208, in some examples, the computing device may provide at least one physiological characteristic to at least one of the first computational model, the second computational model (if any), the third computational model (if any), or the fourth computational model (if any).

At 1210, the computing device may aggregate the output of the first computational model with the outputs of the second computational model, the third computational model, and the fourth computational model, if any.

At 1212, the computing device may receive an output of the aggregation that is indicative of a BP value.

At 1214, the computing device may perform at least one action based on the indicated BP value. For example, the computing device may initiate an alert, and/or may present the BP value on a display associated with the computing device, e.g., as part of a continuous, periodic, or on-demand display of the user's blood pressure. As another example, the computing device may send the BP value to another device to cause the other device to perform an action, such as initiating an alert, presenting the BP value on a display, performing further processing with the BP value, or the like.

The example processes described herein are only examples of processes provided for discussion purposes. Numerous other variations will be apparent to those of skill in the art in light of the disclosure herein. Further, while the disclosure herein sets forth several examples of suitable frameworks, architectures and environments for executing the processes, the implementations herein are not limited to the particular examples shown and discussed. Furthermore, this disclosure provides various example implementations, as described and as illustrated in the drawings. However, this disclosure is not limited to the implementations described and illustrated herein, but can extend to other implementations, as would be known or as would become known to those skilled in the art. Additionally, the implementations herein may be combined with any of the other implementations herein.

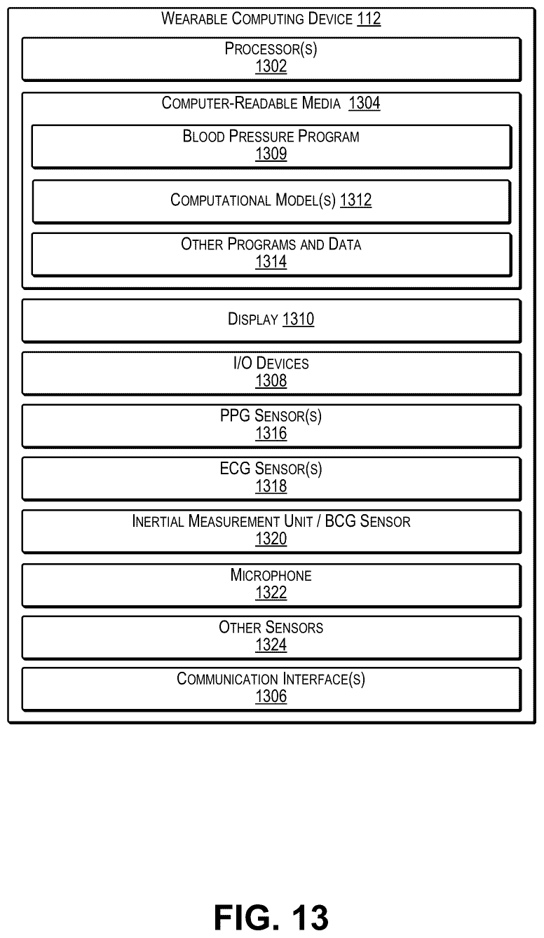

FIG. 13 illustrates select example components of a wearable computing device 112 that may implement the functionality described above according to some examples. The wearable computing device 112 may include any of a number of different types of wearable computing devices and/or body-mounted computing devices, which may include watches, bracelets, wristbands, armbands, gloves, belts, chest straps, anklets, rings, goggles, glasses, helmets, hats, clothing, artificial fingernails, and any other body-mounted device capable of sending and receiving communications and performing the functions according to the techniques and devices described elsewhere herein.

In the example of FIG. 13, the wearable computing device 112 includes components such as at least one processor 1302, one or more computer-readable media 1304, one or more communication interfaces 1306, and one or more input/output (I/O) devices 1308. Each processor 1302 may itself comprise one or more processors or processing cores. For example, the processor 1302 can be implemented as one or more microprocessors, microcomputers, microcontrollers, digital signal processors, central processing units, state machines, logic circuitries, graphics processing units, and/or any devices that manipulate signals based on operational instructions. In some cases, the processor 1302 may be one or more hardware processors and/or logic circuits of any suitable type specifically programmed or configured to execute the algorithms and processes described herein. The processor 1302 can be configured to fetch and execute computer-readable processor-executable instructions stored in the computer-readable media 1304.

Depending on the configuration of the wearable computing device 112, the computer-readable media 1304 may be an example of tangible non-transitory computer storage media and may include volatile and nonvolatile memory and/or removable and non-removable media implemented in any type of technology for storage of information such as computer-readable processor-executable instructions, data structures, program modules, or other data. The computer-readable media 1304 may include, but is not limited to, RAM, ROM, EEPROM, flash memory, solid-state storage, and/or other computer-readable media technology. Further, in some cases, the wearable computing device 112 may access external storage, such as storage arrays, network attached storage, storage area networks, cloud storage, or any other medium that can be used to store information and that can be accessed by the processor(s) 1302 directly or through another computing device or network. Accordingly, the computer-readable media 1304 may be computer storage media able to store instructions, programs, or components that may be executed by the processor(s) 1302. Further, when mentioned herein, non-transitory computer-readable media exclude media such as energy, carrier signals, electromagnetic waves, and signals per se.

The computer-readable media 1304 may be used to store and maintain any number of functional components that are executable by the processor(s) 1302. In some implementations, these functional components comprise instructions or programs that are executable by the processor(s) 1302 and that, when executed, implement operational logic for performing the actions and services attributed above to the wearable computing device 112. Functional components of the wearable computing device 112 stored in the computer-readable media 1304 may include a blood pressure program 1309 that enables the processor(s) 1302 to receive sensor data and determine BP values therefrom. The blood pressure program 1309 may present the BP values on a display 1310 associated with the wearable computing device 112, and/or may communicate the BP values to another computing device, and/or may communicate the sensor data to another computing device, such as discussed above with respect to FIG. 1.

In addition, the computer-readable media 1304 may also store data, data structures, models, and the like, that are used by the functional components. Computer readable media 1304 may store one or more computational models 1312 as discussed above. For instance, as one example, a single computational model 1312 may be employed as a joint model trained for receiving multiple different types of sensor data as inputs, such as discussed above with respect to FIG. 7. In other examples, a plurality of computational models 1312 may be employed, each of which may be trained to receive respective different sensor data such as discussed above with respect to FIG. 8.

Depending on the type of the wearable computing device 112, the computer-readable media 1304 may also optionally include other functional components and data, such as other programs and data 1314, which may include applications, programs, drivers, etc., and the data used or generated by the functional components. Further, the wearable computing device 112 may include many other logical, programmatic, and physical components, of which those described are merely examples that are related to the discussion herein.

The communication interface(s) 1306 may include one or more interfaces and hardware components for enabling communication with various other devices, such as over the network(s) 106 or directly. For example, communication interface(s) 1306 may enable communication through one or more of the Internet, cable networks, cellular networks, wireless networks (e.g., Wi-Fi) and wired networks (e.g., fiber optic, Ethernet), as well as close-range communications such as BLUETOOTH®, BLUETOOTH® low energy, and the like.

FIG. 13 further illustrates that the wearable computing device 112 may include the display 1310, which may be any suitable type of electronic display device for visually presenting information, user interfaces, or the like. Alternatively, in some examples, the wearable computing device 112 may not include a display.

The wearable computing device 112 may further include the one or more I/O devices 1308. The I/O devices 1308 may include speakers, a microphone, and various user controls (e.g., buttons, a joystick, a keyboard, a keypad, etc.), a haptic output device, and so forth.

In addition, the wearable computing device 112 may include or may be in communication with a plurality of sensors as discussed above such as PPG sensor(s) 1316, ECG sensor(s) 1318, an IMU and/or BCG sensor(s) 1320, and a microphone 1322. Other components included in the wearable computing device 112 may include various types of other sensors 1324, which may include a satellite positioning system receiver able to receive and indicate location information, as well as various other sensors.

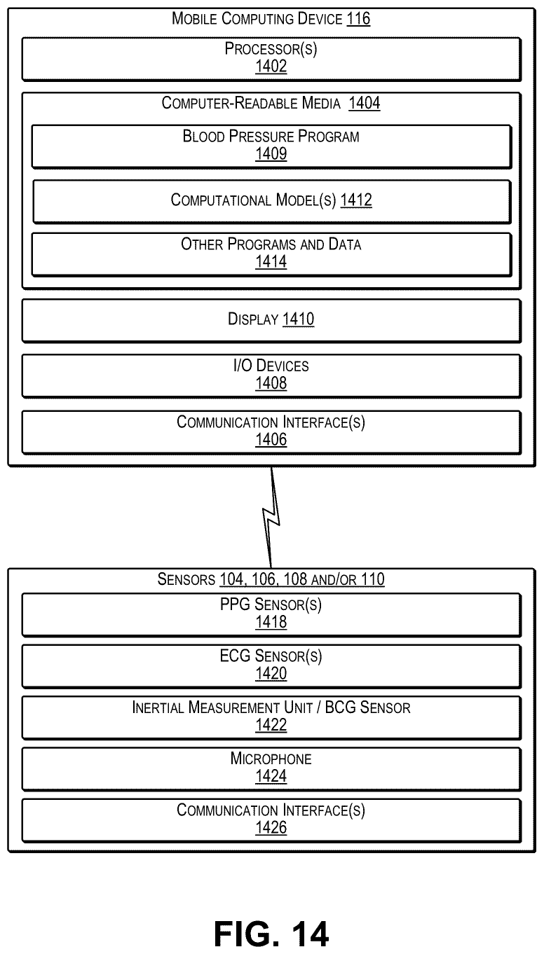

FIG. 14 illustrates select example components of a mobile computing device 116 that may implement the functionality described above according to some examples. The mobile computing device 116 may include any of a number of different types of portable or otherwise mobile computing devices. Some examples of the mobile computing device 116 may include smartphones and mobile communication devices; tablet computing devices; laptops, netbooks and other mobile computers; and any other device capable of sending and receiving communications and performing the functions according to the techniques described herein.

In the example of FIG. 14, the mobile computing device 116 includes components such as at least one processor 1402, one or more computer-readable media 1404, one or more communication interfaces 1406, and one or more input/output (I/O) devices 1408. Each processor 1402 may itself comprise one or more processors or processing cores. For example, the processor 1402 can be implemented as one or more microprocessors, microcomputers, microcontrollers, digital signal processors, central processing units, graphics processing units, state machines, logic circuitries, and/or any devices that manipulate signals based on operational instructions. In some cases, the processor 1402 may be one or more hardware processors and/or logic circuits of any suitable type specifically programmed or configured to execute the algorithms and processes described herein. The processor 1402 can be configured to fetch and execute computer-readable processor-executable instructions stored in the computer-readable media 1404.

Depending on the configuration of the mobile computing device 116, the computer-readable media 1404 may be an example of tangible non-transitory computer storage media and may include volatile and nonvolatile memory and/or removable and non-removable media implemented in any type of technology for storage of information such as computer-readable processor-executable instructions, data structures, program modules, or other data. The computer-readable media 1404 may include, but is not limited to, RAM, ROM, EEPROM, flash memory, solid-state storage, and/or other computer-readable media technology. Further, in some cases, the mobile computing device 116 may access external storage, such as storage arrays, network attached storage, storage area networks, cloud storage, or any other medium that can be used to store information and that can be accessed by the processor(s) 1402 directly or through another computing device or network. Accordingly, the computer-readable media 1404 may be computer storage media able to store instructions, programs, or components that may be executed by the processor(s) 1402. Further, when mentioned herein, non-transitory computer-readable media exclude media such as energy, carrier signals, electromagnetic waves, and signals per se.

The computer-readable media 1404 may be used to store and maintain any number of functional components that are executable by the processor(s) 1402. In some implementations, these functional components comprise instructions or programs that are executable by the processor(s) 1402 and that, when executed, implement operational logic for performing the actions and services attributed above to the mobile computing device 116. Functional components of the mobile computing device 116 stored in the computer-readable media 1404 may include a blood pressure program 1409 that enables the processor(s) 1402 to receive sensor data and determine BP values therefrom. The blood pressure program 1409 may present the BP values on a display 1410 associated with the mobile computing device 116, and/or may communicate the BP values to another computing device, and/or may communicate the sensor data to another computing device, such as discussed above with respect to FIG. 1.

In addition, the computer-readable media 1404 may also store data, data structures, models, and the like, that are used by the functional components. Computer readable media 1404 may store one or more computational models 1412 as discussed above. For instance, as one example, a single computational model may be employed as a joint model that is trained for receiving multiple different types of sensor data as inputs, such as discussed above with respect to FIG. 7. In other examples, a plurality of computational models 1412 may be employed, each of which may be trained to receive respective different sensor data such as discussed above with respect to FIG. 8. Depending on the type of the mobile computing device 116, the computer-readable media 1404 may also optionally include other functional components and data, such as other programs and data 1414, which may include applications, programs, drivers, etc., and the data used or generated by the functional components. Further, the mobile computing device 116 may include many other logical, programmatic, and physical components, of which those described are merely examples that are related to the discussion herein.

The communication interface(s) 1406 may include one or more interfaces and hardware components for enabling communication with various other devices, such as over the network(s) 106 or directly. For example, communication interface(s) 1406 may enable communication through one or more of the Internet, cable networks, cellular networks, wireless networks (e.g., Wi-Fi) and wired networks (e.g., fiber optic, Ethernet), as well as close-range communications such as BLUETOOTH®, BLUETOOTH® low energy, and the like.

FIG. 14 further illustrates that the mobile computing device 116 may include the display 1410, which may be any suitable type of electronic display device for visually presenting information, user interfaces, or the like. Alternatively, in some examples, the mobile computing device 116 may not include a display.

The mobile computing device 116 may further include the one or more I/O devices 1408. The I/O devices 1408 may include speakers, a microphone, and various user controls (e.g., buttons, a joystick, a keyboard, a keypad, etc.), a haptic output device, and so forth. Other components included in the mobile computing device 116 may include various types of sensors (not shown), which may include a satellite positioning system receiver, an accelerometer, gyroscope, compass, proximity sensor, and the like.

The mobile computing device 116 may be in communication with the sensors 104, 106, 108, and/or 110 as discussed above with respect to FIG. 1. For example, the mobile computing device 116 may be able to receive sensor data through a wired or wireless communication with the sensors 104, 106, 108, and/or 110. In this example, the sensors 104, 106, 108, and/or 110 include PPG sensor(s) 1418, ECG sensor(s) 1420, an IMU and/or BCG sensor(s) 1422, and a microphone 1424. The sensors 104, 106, 108, and/or 110 may have a communication interface 1426 associated with them in some examples for communicating the sensor data wirelessly or through a wired connection to the mobile computing device 116. Furthermore, while two example hardware and logical configurations have been discussed herein with respect to FIGS. 13 and 14 numerous other variations will be apparent to those of skill in the art having the benefit of the disclosure herein.

Various instructions, methods, and techniques described herein may be considered in the general context of computer-executable instructions, such as computer programs and applications stored on computer-readable media, and executed by the processor(s) herein. Generally, the terms program and application may be used interchangeably, and may include instructions, routines, modules, objects, components, data structures, executable code, etc., for performing particular tasks or implementing particular data types. These programs, applications, and the like, may be executed as native code or may be downloaded and executed, such as in a virtual machine or other just-in-time compilation execution environment. Typically, the functionality of the programs and applications may be combined or distributed as desired in various implementations. An implementation of these programs, applications, and techniques may be stored on computer storage media or transmitted across some form of communication media.

Although the subject matter has been described in language specific to structural features and/or methodological acts, it is to be understood that the subject matter defined in the appended claims is not necessarily limited to the specific features or acts described. Rather, the specific features and acts are disclosed as example forms of implementing the claims.

What is claimed:

1. A method comprising:
receiving, by a computing device, sensor data from at least one of a photoplethysmographic (PPG) sensor, an electrocardiogram (ECG) sensor, a ballistocardiogram (BCG) sensor, or a microphone;
inputting, by the computing device, the received sensor data into at least one computational model;
receiving, by the computing device, an output of the at least one computational model indicative of a blood pressure (BP) value; and
performing, by the computing device, at least one action based on the indicated BP value.

2. The method as recited in claim 1, wherein the at least one computational model is a joint computational model configured to receive multiple different types of sensor data from multiple different types of sensors, respectively.

3. The method as recited in claim 1, wherein the at least one computational model comprises a first computational model configured to receive the PPG sensor data as an input, and at least one of:
a second computational model configured to receive the ECG sensor data as an input;
a third computational model configured to receive the BCG sensor data as an input; or
a fourth computational model configured to receive the microphone sensor data as an input.

4. The method as recited in claim 3, further comprising aggregating an output of the first computational model with outputs of the at least one of the second computational model, the third computational model, or the fourth computational model to determine the indicated BP value.

5. The method as recited in claim 1, further comprising:
determining, based at least on the sensor data, an indication of at least one physiological characteristic of a portion of a vascular system of a person corresponding to the sensor data; and
providing the at least one physiological characteristic to the at least one computational model.

6. The method as recited in claim 5, wherein the at least one physiological characteristic includes at least one of a resistance or compliance of a portion of an artery of the person.

7. The method as recited in claim 1, wherein performing the at least one action comprises at least one of:
presenting the BP value on a display associated with the computing device;
initiating an alert;
sending the BP value to another device to cause the other device to perform an action, comprising at least one of initiating an alert, displaying the BP value, performing further processing with the BP value.

8. A computing device comprising:
one or more processors configured by executable instructions to perform operations comprising:
receiving, by the one or more processors, sensor data from at least one of a photoplethysmographic (PPG) sensor, an electrocardiogram (ECG) sensor, a ballistocardiogram (BCG) sensor, or a microphone;
inputting, by the one or more processors, the received sensor data into at least one computational model;
receiving, by the one or more processors, an output of the at least one computational model indicative of a blood pressure (BP) value; and
performing, by the one or more processors, at least one action based on the indicated BP value.

9. The computing device as recited in claim 8, wherein the at least one computational model is a joint computational model configured to receive multiple different types of sensor data from multiple different types of sensors, respectively.

10. The computing device as recited in claim 8, wherein the at least one computational model comprises a first computational model configured to receive the PPG sensor data as an input, and at least one of:
a second computational model configured to receive the ECG sensor data as an input;
a third computational model configured to receive the BCG sensor data as an input; or
a fourth computational model configured to receive the microphone sensor data as an input; and
the operations further comprising aggregating an output of the first computational model with outputs of the at least one of the second computational model, the third computational model, or the fourth computational model to determine the indicated BP value.

11. The computing device as recited in claim 8, the operations further comprising:
determining, based at least on the sensor data, an indication of at least one physiological characteristic of a portion of a vascular system of a person corresponding to the sensor data; and
providing the at least one physiological characteristic to the at least one computational model.

12. The computing device as recited in claim 11, wherein the at least one physiological characteristic includes at least one of a resistance or compliance of a portion of an artery of the person.

13. The computing device as recited in claim 8, wherein:
the computing device is a mobile computing device that receives wirelessly the sensor data from the at least one of the PPG sensor, the ECG sensor, the BCG sensor, or the microphone from one or more sensor devices mounted on a person; and
performing, by the one or more processors, the at least one action comprises sending the BP value to at least one wearable computing device included with at least one of the one or more sensor devices mounted on the person.

14. The computing device as recited in claim 8, wherein the computing device is a wearable computing device included in a finger-mountable device, the finger-mountable device including the PPG sensor that provides the PPG sensor data to the one or more processors.

15. A finger-mountable device comprising:
at least one photoplethysmographic (PPG) sensor;
a communication interface; and
one or more processors configured by executable instructions to perform operations comprising:
receiving, by the one or more processors, sensor data from at least the PPG sensor;
inputting, by the one or more processors, the received sensor data into at least one computational model;
receiving, by the one or more processors, an output of the at least one computational model indicative of a blood pressure (BP) value; and
performing, by the one or more processors, at least one action based on the indicated BP value.

16. The finger-mountable device as recited in claim 15, the operations further comprising:
determining, based at least on the sensor data, an indication of at least one physiological characteristic of a portion of a vascular system of a person corresponding to the sensor data; and
providing the at least one physiological characteristic to the at least one computational model, wherein the at least one physiological characteristic includes at least one of a resistance or compliance of a portion of an artery of the person.

17. The finger-mountable device as recited in claim 15, the operations further comprising:
receiving additional sensor data from sensor devices located at one or more other body locations, the additional sensor data being received from at least one of an electrocardiogram (ECG) sensor, a ballistocardiogram (BCG) sensor, or a microphone; and
wherein inputting the PPG sensor data into the at least one computational model, further comprising inputting the additional sensor data into the at least one computational model.

18. The finger-mountable device as recited in claim 15, the operations further comprising sending wirelessly the BP value to a mobile device to cause, at least in part, the mobile device to present the BP value.

19. The finger-mountable device as recited in claim 15, wherein the at least one PPG sensor has a sampling rate greater than 1000 Hz.

20. The finger-mountable device as recited in claim 15, further comprising a display, the operation of performing at least one action comprising presenting the BP value on the display.
